(19) United States
(12) Patent Application Publication
(10) Pub. No.: US 2004/0248915 A1
(43) Pub. Date: Dec. 9, 2004

(54) Method for administration of troxacitabine

(76) Inventors: Jacques Jolivet, Val David (CA); Henriette Gourdeau, Montreal (CA)

Correspondence Address: MILLEN, WHITE, ZELANO & BRANIGAN, PC, 2200 CLARENDON BLVD, SUITE 1400, ARLINGTON, VA, 22201, US

(21) Appl. No.: 10/806,336

(22) Filed: Mar. 23, 2004

Related U.S. Application Data
(60) Provisional application No. 60/465,228, filed on Apr. 25, 2003.

(51) Int. Cl. A61K 31/513

(52) U.S. Cl. 514/269

(57) ABSTRACT

In the treatment of cancer, troxacitabine or a pharmaceutically acceptable salt can be effectively administered to a host having a tumor by continuous infusion for a period of at least 72 hours, wherein said amount is sufficient to provide tumor reduction.

The instant application claims the benefit of U.S. Provisional application Serial No. 60/465,228, filed Apr. 25, 2003, the entire disclosure of which is hereby incorporated by reference.

### FIELD OF THE INVENTION

The present invention relates to methods for treating cancer with troxacitabine, and more particularly, to improvements in the administration of troxacitabine in the treatment of cancer.

### BACKGROUND OF THE INVENTION

Troxacitabine (BCH-4556, Troxatyl™, (−)-β-L-Dioxolane-Cytidine, β-L-OddC) is a nucleoside analogue which was first described as an antiviral agent by Belleau et al. (U.S. Pat. No. 5,041,449). It was also shown that troxacitabine possesed potent antitumor activity and had activity in a broad range of cancers , including in resistant and advanced cancers (K. L. Grove et al., Cancer Res., 55(14), 3008-11, 1995; K. L. Grove et al., Cancer Res., 56(18), 4187-4191, 1996; S. A Kadhim et al., Cancer Res., 57(21), 4803-10, 1997; S. Weitman et al., Clinical Cancer Research, 6, 1574-1578, 2000; H. Gourdeau et al., Cancer Chemother. Pharmacol., 50 490-496, 2002). For example, in clinical studies troxacitabine was reported to have significant activity in patients with primary refractory or relapsed acute myeloid leukemias (AML) (F. J. Giles et al., J. of Clin. Oncology, Vol. 19, No 3, 762-771, 2001; F. J. Giles et al., J. of Clin. Oncology, Vol. 20, No 3, 656-664, 2002).

Unlike naturally occurring nucleosides and current analogues for cancer treatment such as gemcitabine (2′,2′-difluorodeoxycytidine; dFdC) and cytarabine (1-β-D-arabinofuranosylcytosine; araC), which are in the β-D configuration, troxacitabine has a nonnatural β-L configuration which gives rise to unique mechanisms of cellular uptake, metabolism and resistance. For example, Gourdeau et al., in “Mechanisms of Uptake and Resistance to Troxacitabine, a Novel Deoxycytidine Nucleoside Analogue, in Human Leukemic and Solid Tumor Cell Lines”, in Cancer Research, 61, 7217-7224, 2001, reported that membrane permeation was not mediated by nucleoside transporters for troxacitabine, but the major route of cellular uptake was passive diffusion. Troxacitabine also differs from gemcitabine and cytarabine because it is resistant to deamination by cytidine deaminase (CDA). It was also observed that troxacitabine is a complete DNA chain terminator and DNA excision is by apurinic/apyrimidic endonuclease instead of exonucleases (Chou K M et al., J. Biol. Chem., 275: (3), 1009-15, 2000). In addition, it was reported that phosphorylation of troxacitabine from the di-phosphate to the tri-phosphate is by 3-phosphoglycerate kinase instead of nucleoside diphosphate kinase (P. Krishnan et al., J. Biol. Chem., 277 5453-9, 2002).

In Phase I and Phase II clinical trials, it was reported that the recommended doses of troxacitabine with dose-limiting toxicities of neutropenia and skin rash in a study conducted as a 30-minute infusion given on 5 consecutive days every 4 weeks were 1.5 and 1.2 mg/m²/d (J. S. de Bono et al., J. of Clin. Oncology, Vol 20, 96-109, 2002). This corresponds to Cₘₐₓ plasmatic of 0.52-0.66 μM (110-140 ng/ml) and AUC of (1.0-1.31 μM.h) 221-279 ng.h/ml on the first day of injection. On day 5 following injection, a value of Cₘₐₓ of 0.50-0.61 μM (107-129 ng/ml) and AUC of (1.43-1.70 μM.h) 304-362 ng.h/ml is obtained. It was also reported that the recommended doses of troxacitabine with dose-limiting toxicities of neutropenia and skin rash in a study conducted as a 30-minute infusion every 21 days were 12.5 mg/m² and 10 mg/m² (K. Bélanger et al., J. of Clin. Oncology, Vol. 20, No 10, 2567-2574, 2002). This corresponds to Cₘₐₓ of 4.14-4.83 μM (882-1028 ng/ml) and AUC of (8.85-15.52 μM.h) 1886-3306 ng.h/ml. However, in preclinical models it was reported that antitumor efficacy could be observed at doses leading to Cₘₐₓ values much higher than those achievable in humans. The preclinical pharmokinetics of troxacitabine in rats was published in L. F. Moore et al., Cancer Chemother. Pharmacol., Vol 39, 532-536, 1997, and in mice in D. Y. Bouffard et al., Cancer Chemorther. Pharmacol., 52 497-506, 2003 concluding that after a 10 mg/kg (rats) and 20 mg/kg (mice) injection, the Cₘₐₓ obtained was ≧100 μM. Unfortunately, the high Cₘₐₓ values which have shown to be effective in preclinical human xenografts mouse tumor models would be lethal when administered in humans by bolus injections.

Therefore, there is a need to find a new method of administration of troxacitabine that would be more effective compared with the higher bolus doses previously studied in preclinical tumor models. There is also a need to find a new method of administration of troxacitabine that would lead to increased efficacy while not exceeding dose-limiting toxicities.

### SUMMARY OF THE INVENTION

The present invention provides a novel method for the administration of troxacitabine in a host by prolonged continuous infusions of troxacitabine.

In one embodiment, the method provides for the administration of troxacitabine in a host having a tumor, comprising administering troxacitabine by continuous infusion in a low dosage amount, wherein said continuous infusion is by means of a continuous intravenous infusion.

In another embodiment, the method provides for the treatment of cancer within a patient, comprising administering to the patient an effective amount of troxacitabine by continuous infusion for a period of at least 72 hours wherein a steady state plasma concentration of troxacitabine of 0.03 to 2.0 μM is achieved during the administration.

In another embodiment, the method provides for the treatment of cancer within a patient, comprising administering to the patient an effective amount of troxacitabine by continuous infusion for a period of at least 72 hours, wherein the maximum plasma concentration achieved during the administration is 0.03 to 2.0 μM.

In another embodiment, the method provides for the treatment of cancer within a patient, comprising administering to the patient an amount of troxacitabine by continuous infusion for a period of at least 72 hours, wherein the amount is sufficient to provide tumor reduction.

### BRIEF DESCRIPTION OF THE DRAWINGS

Various other features and attendant advantages of the present invention will be more fully appreciated as the same becomes better understood when considered in conjunction with the accompanying drawings, wherein:

FIG. 1 illustrates the results of in vitro assays on troxacitabine cytoxicity using HT-29 human colon carcinoma cells;

FIG. 2 illustrates the results of a study on troxacitabine activity when given as a continuous infusion against the human HT-29 colon tumor xenograft;

FIG. 3 illustrates the results of a study on infusion time for the antitumor efficacy of troxacitabine against the human HT-29 colon tumor xenograft; and

FIG. 4 illustrates the results for comparison study on different adminstration regimens for the administration of troxacitabine.

### DETAILED DESCRIPTION OF THE INVENTION

In one embodiment the method provides for the administration of troxacitabine in a host having a tumor, comprising administering troxacitabine or a pharmaceutically acceptable salt thereof by continuous infusion in a low dosage amount, said amount to be effective to treat the tumor while providing a steady state plasma concentration in the range of 0.03 to 2.0 μM.

In another embodiment the method provides for the treatment of cancer within a patient, comprising administering to the patient an effective amount of troxacitabine or a pharmaceutically acceptable salt thereof by continuous infusion for a period of at least 72 hours wherein a steady state plasma concentration of troxacitabine of 0.03 to 2.0 μM is achieved during the administration.

In another embodiment, the amount of troxacitabine or pharmaceutically acceptable salt thereof administered by continuous infusion provides a steady state plasma concentration in the range of 0.1 to 1.0 μM.

In another embodiment, the amount of troxacitabine or pharmaceutically acceptable salt thereof administered by continuous infusion provides a steady state plasma concentration of 0.05 to 0.1 μM.

In another embodiment, the amount of troxacitabine or pharmaceutically acceptable salt thereof administered by continuous infusion provides a steady state plasma concentration in the range of 0.1 to 0.45 μM (e.g., 0.1 to 0.42 μM).

In another embodiment, a method is provided for the administration of troxacitabine in a host having a tumor, comprising administering troxacitabine by continuous infusion in a low dosage amount, said amount to be effective to treat the tumor while providing a maximum plasma concentration of in the range of 0.03 to 2.0 μM.

In another embodiment the method provides for the treatment of cancer within a patient, comprising administering to the patient an effective amount of troxacitabine or a pharmaceutically acceptable salt thereof by continuous infusion for a period of at least 72 hours wherein the maximum plasma concentration achieved during the administration is 0.03 to 2.0 μM.

In another embodiment, the amount of troxacitabine or pharmaceutically acceptable salt thereof administered by continuous infusion provides a maximum plasma concentration below 1.0 μM.

In another embodiment, the amount of troxacitabine or pharmaceutically acceptable salt thereof administered by continuous infusion provides a maximum plasma concentration below 0.5 μM.

In another embodiment, the amount of troxacitabine or pharmaceutically acceptable salt thereof administered by continuous infusion provides a maximum plasma concentration below 0.45 μM (e.g., below 0.42 μM).

In another embodiment, the amount of troxacitabine or pharmaceutically acceptable salt thereof administered by continuous infusion provides a maximum plasma concentration below 0.1 μM.

In another embodiment the method provides for the treatment of cancer within a patient, comprising administering troxacitabine or a pharmaceutically acceptable salt thereof to the patient by continuous infusion for a period of at least 72 hours at a dose of 0.7 to 12.5 mg/m²/day (e.g., 0.72 to 12.5 mg/m²/day).

In another embodiment, the dosage amount of troxacitabine or pharmaceutically acceptable salt thereof administered by continuous infusion is 1.0 to 11.0 (e.g., 1.2 to 10.1) mg/m²/day.

In another embodiment, the dosage amount of troxacitabine or pharmaceutically acceptable salt thereof administered by continuous infusion is 8.0 to 11.0 (e.g., 8.4 to 10.1) mg/m²/day.

In one embodiment the method provides for the administration of troxacitabine in a host having a tumor, comprising administering troxacitabine by continuous infusion in a low dosage amount, said amount to be effective to provide tumor reduction.

In one embodiment the method provides for the administration of troxacitabine in a host having a tumor, comprising administering troxacitabine by continuous infusion in a low dosage amount, said amount to be effective to treat the tumor while producing fewer side effects.

In one embodiment the method provides for the administration of troxacitabine in a host having a tumor, comprising administering troxacitabine by continuous infusion in a low dosage amount, said amount to be effective to treat the tumor while producing less disruption to the host having said tumor than a prolonged, repeated bolus injection regimen.

In another embodiment the method provides for the administration of troxacitabine in a host having a tumor, comprising administering troxacitabine by continuous infusion in a low dosage amount, said amount to be effective to treat the tumor while not exceeding dose limiting toxicities.

In another embodiment, the method provides the administration of troxacitabine, as described above, in which said continuous infusion is administered for a period of 3 to 7 days.

In another embodiment, the method provides the administration of troxacitabine, as described above, in which said continuous infusion is administered for a period of 3 days.

In another embodiment, the method provides the administration of troxacitabine, as described above, in which said continuous infusion is administered for a period of 4 days.

In another embodiment, the method provides the administration of troxacitabine, as described above, in which said continuous infusion is administered for a period of 5 days.

In another embodiment, the method provides the administration of troxacitabine, as described above, in which said continuous infusion is administered for a period of 6 days.

In another embodiment, the method provides the administration of troxacitabine, as described above, in which said continuous infusion is administered for a period of 7 days.

In one embodiment, the method provides for the administration of troxacitabine in a host having a tumor, comprising administering troxacitabine by continuous infusion in a low dosage amount, wherein said continuous infusion is by means of continuous intravenous infusion.

In another embodiment the method provides for the administration of troxacitabine in a host having a tumor, comprising administering troxacitabine by continuous infusion in a dosage amount of troxacitabine of 9.5 to 10.5 mg/m²/day, said amount to be effective to treat the tumor.

In another embodiment the method provides for the administration of troxacitabine in a host having a tumor, comprising administering troxacitabine by continuous infusion in a dosage amount of troxacitabine of 9.5 to 10.5 mg/m²/day for 4 days, said amount to be effective to treat the tumor while providing a steady state plasma concentration in the range of 0.03 to 2.0 μM.

In another embodiment the method provides for the administration of troxacitabine in a host having a tumor, comprising administering troxacitabine by continuous infusion in a dosage amount of troxacitabine of 9.5 to 10.5 mg/m²/day for 5 days, said amount to be effective to treat the tumor while providing a steady state plasma concentration in the range of 0.03 to 2.0 μM.

In another embodiment the method provides for the administration of troxacitabine in a host having a tumor, comprising administering troxacitabine by continuous infusion in a dosage amount of troxacitabine of 9.5 to 10.5 mg/m²/day for 6 days, said amount to be effective to treat the tumor while providing a steady state plasma concentration in the range of 0.03 to 2.0 μM.

In another embodiment the method provides for the administration of troxacitabine in a host having a tumor, comprising administering troxacitabine by continuous infusion in a dosage amount of troxacitabine of 9.5 to 10.5 mg/m²/day for 7 days, said amount to be effective to treat the tumor while providing a steady state plasma concentration in the range of 0.03 to 2.0 μM.

In another embodiment, the method provides the administration of troxacitabine, as described above, further comprising repeating the period of continuous infusion at an interval of every 4 weeks.

In another embodiment, the method provides the administration of troxacitabine, as described above, further comprising repeating the period of continuous infusion at an interval of every 3 weeks.

In another embodiment, the method provides the administration of troxacitabine, as described above, further comprising repeating the period of continuous infusion at an interval of every 5 weeks.

In another embodiment, there is provided a method for the administration of troxacitabine in a host by prolonged continuous infusions of troxacitabine as described above, in combination with at least one further therapeutic agent selected from the group comprising nucleoside analogues; chemotherapeutic agents; multidrug resistance reversing agents; and biological response modifiers.

In another embodiment, the chemotherapeutic agents are selected from the group consisting of Asparaginase, Bleomycin, Busulfan, Carmustine, Chlorambucil, Cladribine, Cyclophosphamide, Cytarabine, acarbazine, Daunorubicin, Doxorubicin, Etoposide, Fludarabine, Gemcitabine, Gleevec®, Hydroxyurea, Idarubicin, Ifosfamide, Lomustine, Mechlorethamine, Melphalan, Mercaptopurine, Methotrexate, Mitomycin, Mitoxantrone, Pentostatin, Procarbazine, 6-Thioguanine, Topotecan, Vinblastine, Vincristine, Dexamethasone, Retinoic acid and Prednisone.

In one embodiment, the multidrug resistance reversing agent is PSC 833.

In another embodiment, the biological response modifiers are selected from the group consisting of monoclonal antibodies and cytokines.

In another embodiment, the cytokines are selected from the group consisting of interferons, interleukins and colony-stimulating factors.

In another embodiment, the biological response modifiers are selected from the group consisting of Rituxan, CMA-676, Interferon-alpha recombinant, Interleukin-2, Interleukin-3, Erythropoetin, Epoetin, G-CSF, GM-CSF, Filgrastim, Sargramostim and Thrombopoietin.

The individual components of such combinations may be administered either sequentially or simultaneously in separate or combined pharmaceutical formulations.

The combinations referred to above may conveniently be presented for use in the form of a pharmaceutical formulation and thus pharmaceutical formulations comprising a combination as defined above together with an acceptable carrier therefor comprise a further aspect of the invention.

In one embodiment the present invention provides a method for treating cancer selected from the group comprising lung cancer, prostate cancer, renal cancer, hepatoma, bladder cancer, colorectal cancer, pancreatic cancer, gastric cancer, breast cancer, ovarian cancer, soft tissue sarcoma, osteosarcoma, hepatocellular carcinoma, skin cancer, leukemia and lymphomas in patients.

According to a further embodiment, the present invention provides a method for treating pancreatic cancer.

In one embodiment the present invention provides a method for treating leukemia selected from the group comprising acute myelogenous leukemia (AML), chronic myelogenous leukemia (CML), chronic myelogenous leukemia in blastic phase (CML-BP), refractory myelodysplastic syndromes (MDS).

According to a further embodiment, the present invention provides a method for treating pancreatic, renal and acute myelogenous leukemia.

Still in another embodiment, the present invention provides a novel method for treating multidrug resistant cancers.

There is also provided a method for treating leukemia with the pharmaceutically acceptable salts of troxacitabine. By the term pharmaceutically acceptable salts of troxacitabine are meant those derived from pharmaceutically acceptable inorganic and organic acids and bases. Examples of suitable acids include hydrochloric, hydrobromic, sulphuric, nitric, perchloric, fumaric, maleic, phosphoric, glycollic, lactic, salicylic, succinic, toluene-p-sulphonic, tartaric, acetic, citric, methanesulphonic, formic, benzoic, malonic, naphthalene-2-sulphonic and benzenesulphonic acids.

Salts derived from appropriate bases include alkali metal (e.g. sodium), alkaline earth metal (e.g. magnesium), ammonium and NR₄₊(where R is C₁₋₄ alkyl) salts.

The term “Cₘₐₓ” represents the maximum plasma concentration.

The term “steady state plasma concentration” as applied to troxacitabine, is the stable plasma concentration which is achieved after an infusion length of at least 5 half-lives. As per the article of K. Bélanger et al., J. of Clin. Oncology, Vol. 20, No 10, 2567-2574, 2002, the steady state plasma concentration of troxacitabine is achieved in 60 hrs (5×12 hrs) or 2½ days.

The term “dose limiting toxicity” (DLT) represents toxicities for troxacitabine which include neutropenia, hand-foot syndrome, stomatitis and skin rash. More specifically, DLT's for leukemia with troxacitabine represent grade III and grade IV non-hematologic toxicities, for eg., hand-foot syndrome and stomatitis. More specifically, DLT's for solid tumors with troxacitabine represent grade III and grade IV hematologic and non-hematologic toxicities, for eg., neutropenia, hand-foot syndrome and stomatitis.

The term “host” represents any mammals including humans.

The term “leukemia” represent acute myelogenous leukemia (AML), chronic myelogenous leukemia (CML), chronic myelogenous leukemia in blastic phase (CML-BP), acute lymphocytic leukemia (ALL), chronic lymphocytic leukemia (CLL), hairy cell leukemia (HCL), myelodysplastic syndromes (MDS) and all subtypes of these leukemias which are defined by morphological, histochemical and immunological techniques that are well known by those skilled in the art.

The term “multidrug resistant cancer” represents a cancer which is non responsive to treatment with two or more chemotherapeutic agents.

The term “solid tumor” represents a cancer selected from the group comprising lung cancer, prostate cancer, renal cancer, hepatoma, bladder cancer, colorectal cancer, pancreatic cancer, gastric cancer, breast cancer, ovarian cancer, soft tissue sarcoma, osteosarcoma, hepatocellular carcinoma and skin cancer in patients.

The term “tumor reduction” represents in leukemia the reduction of blast cells in the blood and the reduction of blast cells in the bone marrow. The term “tumor reduction” in solid tumors represents the reduction of tumor mass either by radiographic exam, including PET and NMR scans or by physical examination.

In one embodiment, the host is human.

It will be appreciated that the amount of a troxacitabine of the present invention required for use in treatment will vary with the route of administration, the nature of the condition for which treatment is required and the age and condition of the patient and will be ultimately at the discretion of the attendant physician or veterinarian. In general, however, a suitable dose will be in the range of from about 0.72 to about 12.5 mg/m²/day. For example, a suitable dose can be in the range of from about 1.0 to about 11.0 mg/m²/day, such as about 1.2 to about 10.1 mg/m²/day. A suitable dose for the treatment of leukemia can be in the range of, for example, from about 8 to about 10.0 mg/m²/day, such as about 8.4 to about 10.1 mg/m²/day. In a further embodiment, a suitable dose in the treatment of leukemia can be, for example, from about 9.5 to about 10.0 mg/m²/day, such as 10.1 mg/m²/day. A suitable dose in the treatment of solid tumors can be, for example, in the range of from about 2.0 to about 3.0 mg/m²/day. In a further embodiment, a suitable dose in the treatment of solid tumors can be, for example, about 2.0 to about 2.5 mg/m²/day, such as 2.25 mg/m²/day.

Ideally, the active ingredient is administered to achieve peak plasma concentrations of the active compound of from about 0.03 to about 2 μM, preferably at about to about 0.4 to 2 μM, most preferably about 0.1 to about 0.42 μM. This may be achieved, for example, by continuous infusion containing about 1 to about 500 mg of the active ingredient. Desirable blood levels may be maintained by a continuous infusion to provide about 0.01 to about 5.0 mg/kg/hour or by infusions containing about 0.4 to about 15 mg/kg of the active ingredient.

While it is possible that, for use in therapy, troxacitabine may be administered as the raw chemical, it is preferable according to one embodiment of the invention, to present the active ingredient as a pharmaceutical formulation. The embodiment of the invention thus further provides a pharmaceutical formulation comprising troxacitabine or a pharmaceutically acceptable salt thereof together with one or more pharmaceutically acceptable carriers therefor and, optionally, other therapeutic and/or prophylactic ingredients. The carrier(s) must be “acceptable” in the sense of being compatible with the other ingredients of the formulation and not deleterious to the recipient thereof.

Infusions are parenteral products intended for injection into a vein by intravenous (i.v.) drip, as further described in Pharmaceutical Dosage Forms: Parenteral Medications, Vol 1, Ed. Kenneth E. Avis, Herbert A. Lieberman and Leon Lachman, 2^{nd} Ed., 1992, pp514-518. Furthermore, they are packaged in plastic or glass large volume parenteral (LVP) containers to which is attached a sutitable i.v. set at the time of infusion. Venous entry is by a metal needle or a plastic catheter.

A continuous infusion system provides continuous, regulated, fluid flow at a preset rate. Once a prescribed flow rate (e.g., 125 ml/hr) has been established, the fluid should continue to flow accurately from the system until the container has emptied. Fluids are infused according to a continuous or an intermittent dose schedule. A continuous schedule involves the nonstop infusion of a relatively large volume of fluid (e.g., 1 liter per 8 hr period for adults) over a period of at least 3 days. Continuous therapy usually provides fluid, electrolytes, agents to adjust acid-base balance, nutrients and some drugs by slow i.v. drip. The total fluid intake must not exceed the patient's requirements, approximately 2400 ml for an adult.

Infusions are administered with or without additives. The preparation of i.v. admixtures constitutes by far the predominant involvement of the hospital pharmacist in sterile pharmaceutical compounding.

Troxacitabine according to the present invention may be formulated for parenteral administration by injection, for example continuous infusion) and may be presented in unit dose form in ampoules, pre-filled syringes, small volume infusion or in multi-dose containers with an added preservative. The compositions may take such forms as suspensions, solutions, or emulsions in oily or aqueous vehicles, and may contain formulatory agents such as suspending, stabilizing and/or dispersing agents.

The following examples are provided to illustrate various embodiments of the present invention and shall not be considered as limiting in scope.

In the foregoing and in the following examples, all temperatures are set forth uncorrected in degrees Celsius; and, unless otherwise indicated, all parts and percentages are by weight.

Troxacitabine (β-L OddC) was synthesized at Shire BioChem Inc. as previously described in PCT publication numbers WO96/07413A1, WO97/21706 and WO00/47759.

### EXAMPLE 1

### Time and Concentration Dependent in Vitro Cytotoxicity Assays

In order to define troxacitabine's concentration and exposure requirements for optimal efficacy, these two parameters were examined using the HT-29 human colorectal carcinoma cell line. Two different methods were used, the MTT colometric assay and the clonogenic assay.

a)MTT colometric assay: HT-29 cells were plated at 2.5×10³ cells/well in a 96-well tissue culture plate and allowed to attach overnight. The next day (day 0), troxacitabine was added to the culture medium for 1, 4, 24 or 72 hr. After the incubation period with the drug, cells were washed and fresh drug-free medium added. On day 3, cellular proliferation was evaluated by the tetrazolium dye assay.

b)Clonogenic assay: HT-29 cells were plated on 6-well flat-bottomed plates (200-400 cells) and allowed to attach overnight. The next day (day 0), troxacitabine was added to the culture medium for 24, 72 or 144 hr. After the incubation period with the drug, cells were washed and fresh drug-free medium added. On day 10, cells were exposed to Crystal violet stain. Colonies greater than 50 cells were counted.

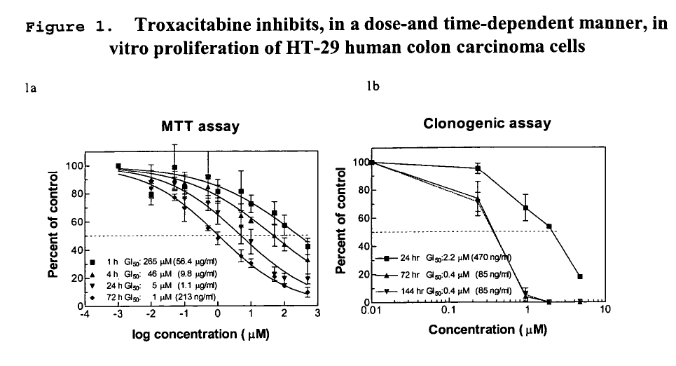

As shown in FIG. 1a, troxacitabine cytotoxic activity is concentration and time dependent. Troxacitabine showed weak inhibitory activity when drug exposure was short (1 hr and 4 hr) with GI₅₀ values of 265 and 46 μM, respectively, with 72-hour being 265-fold more potent than 1 hr exposures. Longer drug exposures were examined in the clonal growth assay, as shown in FIG. 1b. The 5-fold difference between IC₅₀ values observed following 24-and 72 hr exposures (2.2 and 0.4 μM, respectively) was similar to that observed in the MTT assay, where GI₅₀ values of 5 and 1 μM were obtained, respectively. No further increase in potency was found when incubation time was increased from 72 hr to 144 hr.

### EXAMPLE 2

### Schedule-dependent in Vivo Efficacy Studies in Human Xenografts

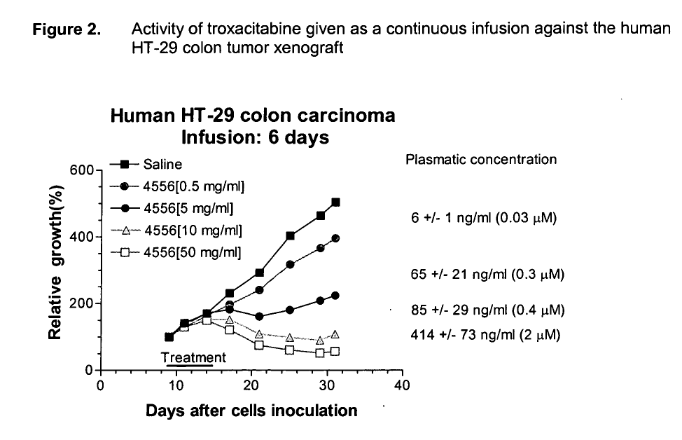

Antitumor efficacy studies: In vivo efficacy studies were conducted using the HT-29 human colorectal cancer cells grown as xenografts in nude mice. Female athymic CD-1(nu/nu) mice were injected s.c. with 2×106 human colon HT-29 tumor cells. Tumor bearing animals were randomised (10 to 15 per group) and treatment started when tumor volumes reached 80 to 120 mm³ (days 6 to 9; depending on the study). Tumor measurements, taken by callipers twice weekly, were converted to tumor volumes (in mm³) using the standard formula, [width (mm)]²×length (mm)×0.52. Troxacitabine treatments were performed either by single or multiple bolus i.v. injections (q1dx5 or q7dx3) or by continuous administration via Alzet® osmotic minipumps (ALZA, Palo Alto. Calif.). Troxacitabine doses are presented in the FIGS. 2, 3 and 4. The pumps were implanted s.c. under sterile conditions at the site opposite to the tumor. These pumps (internal volume, 200 μl) continuously deliver test agent at a rate of 1 μl/h for 200 h (8.3 days total). Pumps were removed after 1, 3, 5 or 6 days, thus insuring continuous delivery of test agent over the course of the experiment. The control groups received comparable bolus injection of saline (i.v.) or s.c. implanted saline-loaded pumps. Compound efficacy was assessed at the end of the study by percentage of T/C defined as the mean treated tumor volume/mean control tumor volume×100%, with a T/C of about 40% or less being indicative of antitumor activity (J. Plowman et al., Human tumor xenograft models in NCI drug development in Teicher BA (ed) “Anticancer Drug Development Guide: Preclinical screening, clinical trials, and approval”. Humana Press Inc., Totowa, N.J. 1997;101-125). To monitor the drug-associated toxicity, mice were weighed at least twice a week and daily inspected for observable clinical signs. Statistical analysis was performed by ANOVA (ANalysis Of VAriance) or by Student's t test. Differences were considered to be significant at P<0.05.

In a first study, troxacitabine was administered as a continuous infusion using Alzet® minipumps at doses of 0.5, 5, 10 and 50 mg/ml for 6 consecutive days, when the tumors had reached 100-120 mm³ (day 9)as shown in FIG. 2.

The 50 mg/ml dose, equivalent to 45-50 mg/kg/day, was considered as the maximum tolerated dose (MTD) since we observed ≦20% body weight loss 3-5 days following initiation of treatment. Furthermore, one mouse, from this group, had to be sacrificed due to significant weight loss. The remaining mice from this group started to gain weight at day 17, and by day 26 their body weight had reached those of the control animals. Body weight gain of mice from the other treated groups was comparable to those from the saline treated group. Animals were sacrificed on day 31, time at which the tumor volumes in the saline treated control group had reached 5 times their original volume (500% growth). T/C's were 79%, 42%, 22% and 11% for the 0.5, 5, 10 and 50 mg/ml doses, respectively, indicating antitumor activity at all but the lower dose level. Moreover, tumor regressions were observed from days 29 and 21 in animals receiving 10 and 50 mg/ml of troxacitabine, respectively (FIG. 2).

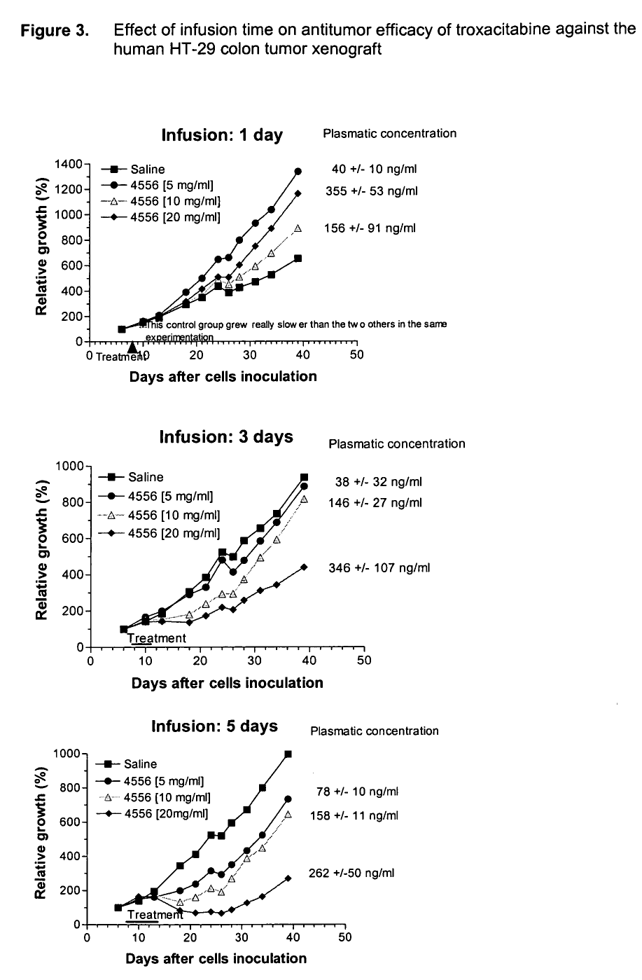

Once we had established the optimum dose range (activity and well tolerated toxicity), a second study was performed to establish the time necessary to obtain optimum antitumor efficacy. This study was performed using 5, 10 and 20 mg/ml infused over 1, 3 and 5 days, when the tumors had reached 85 mm³ (day 6). As shown in FIG. 3, the one day infusion schedule (24 h), irregardless of the dose, had no effect and tumor growth was similar in all treated groups (FIG. 3, upper panel). Antitumor activity was observed with 20 mg/ml when infused over 3 days (FIG. 3, middle panel). This dose and schedule resulted in tumor stasis during the first week after treatment followed by tumor regrowth. At the end of the study, when tumors in the control group had increased by 9 to 10-fold, the T/C was 44% (P value of 0.03 when compared to the saline treated group). This dose resulted in significant antitumor activity when given over a 5-day infusion period (FIG. 3, lower panel). Tumor regressions were observed from day 18 till day 28 followed by regrowth, leading to a T/C of 27%. This data indicated that troxacitabine had potent antitumor activity at well tolerated doses administered continuously for 5 days.

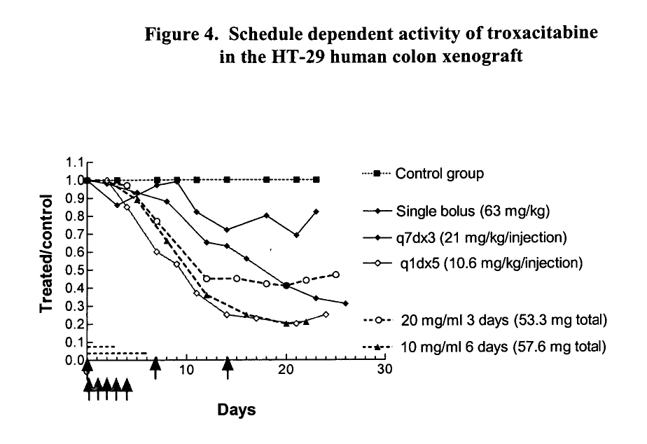

In a third study (FIG. 4), we compared the antitumor activity of troxacitabine given by continuous infusion to that of single and repeated bolus administration using similar doses (53-63 mg/kg). Bolus administration was given by intravenous injection in the tail vein (10 ml/kg). As can be seen from FIG. 4, a single bolus injection of a high dose (63 mg/kg) showed low activity, inhibition of tumor growth was intermediate with 3-day administration regimens (q7d×3 or 3-day infusions) and optimal with 5-6 day regimens (q1d×5 or 6-day infusions). Repeated bolus administration (q1dx5) and prolonged drug administration (continuous infusions of 3 days and more) led to superior antitumour activity compared to a large single bolus administration.

### EXAMPLE 3

### Comparison of Murine vs. Human Pharmacokinetics

This study compared the pharmacokinetics of troxacitabine in nude mice with the same parameters achieved in humans in previous clinical studies. As reported in D. Y. Bouffard et al., Cancer Chemother. Pharmacol., 52 497-506, 2003, following a bolus i.v. dose of 20 mg/kg, the troxacitabine plasma concentration taken at 2 minutes (earliest time point and considered as the Cₘₐₓ) was ≧100 μM with an elimination t_{1/2} of 16 minutes. By 60 minutes, drug concentration was down to 4.5 μM, which is well above the IC₅₀ value for human cells (FIG. 1). By comparison, following i.v. administration of troxacitabine at 10 mg/m² (maximum tolerated dose in humans), the troxacitabine plasma Cₘₐₓ was 4.1 μM with an elimination t_{1/2} of 13 hours, as reported in Bélanger et al., 2002, J. Clin. Oncol. Less than 30 minutes after the end of infusion, drug concentration was down to less than 1 μM but it remained above 0.1 μM for approximately 12 hours. Mice can thus tolerate much higher drug systemic concentrations (over 20-fold) and exposures (over 6-fold) compared with humans.

To determine plasma concentrations of troxacitabine following continuous infusion, blood samples from mice receiving continuous administration via mini-osmotic pumps containing 5, 10 and 20 mg/ml of troxacitabine (n=5/time point) were collected at 3 hours, 1 day, 3 days and 5 days after troxacitabine-loaded pump implantation. Blood was centrifuged at 10,000 g for 10 minutes and plasma was collected and frozen at −20° C. until further analysis. An aliquot of mouse plasma (100 μl) was precipitated with 300 μl of acetonitrile containing 3TC® (Shire BioChem) as internal standard. After centrifugation at 3,000 ×g for 5 minutes, 300 μl of supernatant was collected and evaporated to dryness under a gentle stream of nitrogen. The residue was then reconstituted with 100 μl of deionized water. The calibration curve of troxacitabine was linear between 1 and 500 ng/ml. The chromatography was achieved on a Luna C18 column (150×2 mm, 5 μm, Phenomenix, Torrance, Calif.) with a flow rate of 0.25 ml/min. The aqueous mobile phase, consisting of 10 mM ammonium acetate buffer, pH6.8, was held for 5 minutes and followed by a linear gradient of 0-50% acetonitrile over 8 minutes. Sample analysis was performed on a LC/MS/MS (TSQ7000, Thermo Finnigan, San Jose, Calif.) equipped with electrospray source. The signals of troxacitabine and the internal standard were monitored by m/z transitions at 214- 112 and 230- 112, respectively.

During continuous s.c. infusion using Alzet® osmotic minipumps containing 10 or 20 mg/ml, mean troxacitabine steady state values of 152 ng/ml (0.7 μM) and 304 ng/ml (1.4 μM) were achieved by three hours after pump implantation and maintained over the experimental study (data for the 1, 3 and 5 day exposure is presented in FIG. 2 and FIG. 3). Continuous administration of troxacitabine at 10 mg/ml thus resulted in a total exposure of 10.5 and 19 μg*h/ml when administered over 3 and 5 days, respectively. These values were 24.9 and 31.4 μg*h/ml following the 20 mg/ml administration.

Conclusions: From the pharmokinetic comparison studies, it can be seen that mice can tolerate high dose bolus drug administration yielding plasma concentrations of ≧100 μM 2 minutes after drug administration. However, a similar bolus dose administration strategy has not been achievable in humans, as described in Bélanger et al., J. Clin. Oncol. 2002, 20, 2567-2574; De Bono et al., J. Clin. Oncol. 2002, 20, 96-109; Canova et al., J. Clin. Oncol. 1999, 20, 2567-2574; Giles et al., J. Clin. Oncol. 2001, 19, 762-771. As documented in (paper in preparation), human tissues phosphorylate troxacitabine much more readily than murine tissues and are thus significantly more sensitive to troxacitabine's toxic effects. Administered clinical doses have thus been proportionally over 15 to 125-fold lower than those used in nude mice yielding plasma concentrations of 0.6 to 4 μM 5 minutes after drug administration. Consequently, the drug concentration profiles observed in human plasma appear to be sub optimal to achieve a maximal therapeutic effect based on the preclinical in vitro and in vivo data.

As reported in the above human xenograft studies, prolonged exposures to low micromolar troxacitabine concentrations (0.7-1.6 μM), lead to the same tumor growth inhibition as seen following high-dose bolus administration without the need to achieve peak plasma drug concentrations ≧100 μM. Indeed, a continuous infusion of 10 mg/ml over 6 days resulted in the same antitumor activity as a 10.6 mg/kg daily treatment for 5 consecutive days (as can be seen in FIG. 4). While both treatments resulted in similar total doses (57.6 and 53 mg/kg, respectively) they give very different PK profiles resulting in 100 fold differences in peak concentrations (0.7-1.6 μM versus ≧100 μM). Interestingly, total drug exposure of troxacitabine was similar using both regimens. Assuming linearity between drug and plasma concentrations and no accumulation, the 10.6 mg/kg daily bolus treatment for 5 consecutive days would result in an AUC of 18.8 μg*h/ml. In comparison, infusion of 10 mg/ml at a rate of 1μl per hour over 5 consecutive days would result in a calculated AUC of 19 μg*h/ml (for a 24 g mouse). These data suggest that troxacitabine antitumor activity is more dependent on AUC rather than Cₘₐₓ. Furthermore, in order to achieve effective cell kill and optimum antitumor efficacy, exposure needs to be greater than 24 hours (FIG. 1b and FIG. 3).

### Clinical Phase I Studies of Troxacitabine Administered by Continuous Infusion

### EXAMPLE 4

A study is being conducted to evaluate troxacitabine as a continuous infusion in patients with relapsed acute myeloid leukemia (AML). Twenty-four adults with previously treated recurrent AML have been entered so far in this study. Troxacitabine was first administered at a dose infusion rate of 0.42 mg/m²/hr resulting in a targeted delivery of 10.1 mg/m²/day by 24 hour continuous infusion daily for 2, 3, 4, 5 and 6 days in successive cohorts of patients such that cumulative doses ranged from 20.2 g/m² to 60.6 mg/m². Safety data is available for all patients treated so far. Apart from expected grade 4 myelosuppression, which was seen in all patients, troxacitabine-related toxicity primarily included stomatitis, skin rash and hand-foot syndrome. Despite prophylactic prednisone, a generalized mild to moderate skin rash was observed at all dose levels, with only one patient exhibiting grade 3 toxicity at the 40.4 mg/m² dose level. This was considered a dose-limiting toxicity (DLT) and 5 other patients were treated at this dose level without other DLTs, which enabled treating the next cohort at the next highest dose level. Grade 1 and 2 hand-foot syndrome was also observed at all dose levels. No grade 3 hand-foot syndrome was seen after 1 cycle of therapy, but did reach grade 3 in 1 patient receiving a second cycle of troxacitabine. Other toxicities including nausea/vomiting and diarrhea were mild to moderate except for Grade 3 stomatitis, which was dose-limiting for 2 of 4 patients at the 60 mg/m² dose level. The next cohort of patients was treated at a reduced infusion rate (8.4 mg/m² daily) over 6 days for a total dose of 50.4 mg/m². Two of the four patients treated had dose-limiting stomatitis. The recommended dose is thus likely to be 50.5 mg/m² administered over five days, a dose level at which three patients were treated without any DLT. Three additional patients will be treated to ensure its safety. Data on antileukemic activity is available for the first 24 patients treated. Bone marrow hypoplasia/aplasia was not seen in the first 7 patients treated at cumulative doses ≦30.3 mg/m². Bone marrow aplasia was observed in 10 of 17 patients who received 40.4-60.6 mg/m² of troxacitabine. So far, 4 patients achieved remissions. Preliminary pharmacokinetic analysis revealed that the median troxacitabine concentration at the end of the 144-hour infusion at the 10.1 mg/m² infusion rate was 88 ng/mL (approximately 0.4 μM). Interpatient variation in observed concentrations at each protocol time-point varied approximately 2-to 3-fold, with a CV % of approximately 30%. This degree of interpatient variability is consistent with that observed in a previous phase I study where troxacitabine was administered as a 30-min infusion daily for five days. Continuous infusion troxacitabine is well tolerated with 25% higher cumulative doses achieved than by bolus administration and longer drug exposure above or equal to 0.4 μM (120 hrs versus 19.3 hrs, respectively).

### EXAMPLE 5

A study is being conducted to evaluate the toxicity, pharmacokinetics (PK), and antitumor activity of continuous infusion troxacitabine in patients with advanced solid tumors. This is a dose and infusion length finding study to achieve a minimum steady-state plasma concentration of 21 ng/mL (0.1 μM) for 72-120 hr. The starting dose of 3 mg/m²/d was determined from PK simulation modeling using data obtained from Phase I studies (30-minute infusions). The starting infusion duration was 48 hr with escalation to 72 hr, 96 hr and 120 hr in cohorts of 3-6 patients.

In the results evaluated to date, 12 adults with refractory solid tumors have been treated. The first cohort (4 patients) received drug for 48 h (total dose 6 mg/m²) with no DLT. The second cohort of patients was treated for 72 hr (total 9 mg/m²). DLT's were observed in 2 of the 5 patients treated at this dose level consisting of prolonged (>7 d) Grade 4 neutropenia (2 patients) that was complicated with fever (1 patient) and Grade 3 thrombocytopenia (1 patient). This dose level was felt to be too toxic and the next three patients were treated at 2 mg/m²/d administered over 3 days for a total dose of 6 mg/m² with no DLTs observed. The main non-hematological toxicities were mild to moderate skin rash. At 3 mg/m²/d for 48-72 hr, mean (SD) troxacitabine plasma concentrations at 23.5 hr, 47.5 hr, and 71.5 hr were 24 (5.6), 28 (8.0), and 31 (7.2) ng/mL, respectively. The 2 patients with DLT had the highest plasma concentrations. Enrollment is ongoing at 2.25 mg/m²/d for 96 h (total 9 mg/m²).

Conclusion: Preliminary results from the clinical studies suggest that prolonged (up to 5-day) exposures to low micromolar troxacitabine concentrations are clinically feasible. Continued enrolment will continue to define the recommended dose and infusion duration for troxacitabine administered as a continuous infusion both in AML and solid tumor patients.

The entire disclosure of all applications, patents and publications, cited above and below, is hereby incorporated by reference.

The preceding examples can be repeated with similar success by substituting the generically or specifically described reactants and/or operating conditions of this invention for those used in the preceding examples.

From the foregoing description, one skilled in the art can easily ascertain the essential characteristics of this invention and, without departing from the spirit and scope thereof, can make various changes and modifications of the invention to adapt it to various usages and conditions.

1. A method for the treatment of cancer within a patient, comprising administering to said patient an effective amount of troxacitabine or a pharmaceutically acceptable salt thereof by continuous infusion for a period of at least 72 hours wherein a steady state plasma concentration of troxacitabine of 0.03 to 2.0 μM is achieved during the administration.

2. A method according to claim 1, wherein said cancer is lung cancer, prostate cancer, bladder cancer, colorectal cancer, pancreatic cancer, renal cancer, hepatoma, gastric cancer, breast cancer, ovarian cancer, soft tissue sarcoma, osteosarcoma, hepatocellular carcinoma, skin cancer, leukemia or lymphomas.

3. A method according to claim 2, wherein said cancer is pancreatic cancer.

4. A method according to claim 1, wherein said cancer is acute myelogenous leukemia, chronic myelogenous leukemia, chronic myelogenous leukemia in blastic phase, or refractory myelodysplastic syndromes.

5. A method according to claim 4, wherein said cancer is acute myelogenous leukemia.

6. A method according to claim 2, wherein a steady state plasma concentration of 0.05 to 0.1 μM is achieved during the administration.

7. A method according to claim 4, wherein a steady state plasma concentration of 0.1 to 0.42 μM is achieved during the administration.

8. A method for the treatment of cancer within a patient, comprising administering to said patient an effective amount of troxacitabine or a pharmaceutically acceptable salt thereof by continuous infusion for a period of at least 72 hours, wherein the maximum plasma concentration achieved during the administration is 0.03 to 2.0 μM.

9. A method according to claim 8, wherein the maximum plasma concentration achieved during the administration is below 1.0 μM.

10. A method according to claim 8, wherein the maximum plasma concentration achieved during the administration is below 0.5 μM.

11. A method according to claim 8, wherein the maximum plasma concentration achieved during the administration is below 0.42 μM.

12. A method according to claim 8, wherein the maximum plasma concentration achieved during the administration is below 0.1 μM.

13. A method for the treatment of cancer within a patient, comprising administering to said patient troxacitabine or a pharmaceutically acceptable salt thereof by continuous infusion for a period of at least 72 hours at a dose of 0.72 to 12.5 mg/m²/day.

14. A method according to claim 13, wherein the dosage amount of troxacitabine or a pharmaceutically acceptable salt thereof is 1.0 to 11.0 mg/m²/day.

15. A method according to claim 13, wherein the dosage amount of troxacitabine or a pharmaceutically acceptable salt thereof is 8.0 to 11.0 mg/m²/day.

16. A method according to claim 13, wherein said cancer is lung cancer, prostate cancer, bladder cancer, colorectal cancer, renal cancer, hepatoma, pancreatic cancer, gastric cancer, breast cancer, ovarian cancer, soft tissue sarcoma, osteosarcoma, hepatocellular carcinoma, skin cancer, leukemia or lymphoma.

17. A method according to claim 16, wherein said cancer is pancreatic cancer.

18. A method according to claim 13, wherein said cancer is acute myelogenous leukemia, chronic myelogenous leukemia, chronic myelogenous leukemia in blastic phase, or refractory myelodysplastic syndromes.

19. A method according to claim 18, wherein said cancer is acute myelogenous leukemia.

20. A method according to claim 16, wherein the dosage amount of troxacitabine or a pharmaceutically acceptable salt thereof is 2.0 to 3.0 mg/m²/day.

21. A method according to claim 13, wherein said cancer is a solid tumor and the dosage amount of troxacitabine or a pharmaceutically acceptable salt thereof is 2.0 to 2.5 mg/m²/day.

22. A method according to claim 18, wherein the dosage amount of troxacitabine or a pharmaceutically acceptable salt thereof 9.5 to 10.5 mg/m²/day.

23. A method according to claim 1, wherein said continuous infusion is administered for a period of 3 to 7 days.

24. A method according to claim 1, wherein said continuous infusion is administered for a period of 3 days.

25. A method according to claim 1, wherein said continuous infusion is administered for a period of 4 days.

26. A method according to claim 1, wherein said continuous infusion is administered for a period of 5 days.

27. A method according to claim 1, wherein said continuous infusion is administered for a period of 6 days.

28. A method according to claim 1, wherein said continuous infusion is administered for a period of 7 days.

29. A method according to claim 16, wherein the dosage amount of troxacitabine or a pharmaceutically acceptable salt thereof is 2.0 to 2.5 mg/m²/day, said period is 3 days, and a steady state plasma concentration of 0.05 to 0.1 μM of troxacitabine or a pharmaceutically acceptable salt thereof is achieved during the administration.

30. A method according to claim 16, wherein the dosage amount of troxacitabine or a pharmaceutically acceptable salt thereof is 2.0 to 2.5 mg/m²/day, said period is 4 days, and a steady state plasma concentration of 0.05 to 0.1 μM of troxacitabine or a pharmaceutically acceptable salt thereof is achieved during the administration.

31. A method according to claim 18, wherein the dosage amount of troxacitabine or a pharmaceutically acceptable salt thereof is 9.5 to 10.5 mg/m²/day, said period is 5days, and a steady state plasma concentration of 0.1 to 0.42 μM of troxacitabine or a pharmaceutically acceptable salt thereof is achieved during the administration.

32. A method according to claim 18, wherein the dosage amount of troxacitabine or a pharmaceutically acceptable salt thereof is 9.5 to 10.5 mg/m²/day, said period is 6 days, and a steady state plasma concentration of 0.1 to 0.42 μM of troxacitabine or a pharmaceutically acceptable salt thereof is achieved during the administration.

33. A method according to claim 1, further comprising repeating said continuous infusion at an interval of every 4 weeks.

34. A method according to claim 1, further comprising repeating said continuous infusion at an interval of every 3 weeks.

35. A method according to claim 1, further comprising repeating said continuous infusion at an interval of every 5 weeks.

36. A method according to claim 1, wherein said continuous infusion is by means of continuous intravenous infusion.

37. A method according to claim 1, wherein said method further comprising, in combination with said continuous administration of troxacitabine, administering at least one further therapeutic agent selected from the group comprising nucleoside analogues; chemotherapeutic agents; multidrug resistance reversing agents; and biological response modifiers.

38. A method according to claim 37, wherein said at least one further therapeutic agent is a chemotherapeutic agent selected from Asparaginase, Bleomycin, Busulfan, Carmustine, Chlorambucil, Cladribine, Cyclophosphamide, Cytarabine, Dacarbazine, Daunorubicin, Doxorubicin, Etoposide, Fludarabine, Gemcitabine, Gleevec®, Hydroxyurea, Idarubicin, Ifosfamide, Lomustine, Mechlorethamine, Melphalan, Mercaptopurine, Methotrexate, Mitomycin, Mitoxantrone, Pentostatin, Procarbazine, 6-Thioguanine, Topotecan, Vinblastine, Vincristine, Dexamethasone, Retinoic acid and Prednisone.

39. A method according to claim 37, wherein said at least one further therapeutic agent is the multidrug resistance reversing agent PSC 833.

40. A method according to claim 37, wherein said at least one further therapeutic agent is a biological response modifier selected from monoclonal antibodies and cytokines.

41. A method according to claim 37, wherein said at least one further therapeutic agent is a cytokine selected from interferons, interleukins and colony-stimulating factors.

42. A method according to claim 37, wherein said at least one further therapeutic agent is a biological response modifier selected from Rituxan, CMA-676, Interferon-alpha recombinant, Interleukin-2, Interleukin-3, Erythropoetin, Epoetin, G-CSF, GM-CSF, Filgrastim, Sargramostim and Thrombopoietin.

43. A method according to claim 37 wherein said of troxacitabine or a pharmaceutically acceptable salt thereof and said at least one further therapeutic agent are administered sequentially.

44. A method according to claim 37 wherein said of troxacitabine or a pharmaceutically acceptable salt thereof and at least one further therapeutic agent are administered simultaneously.

45. A method according to claim 44 wherein said of troxacitabine or a pharmaceutically acceptable salt thereof and at least one further therapeutic agent are administered in separate pharmaceutical formulations.

46. A method according to claim 44 wherein said of troxacitabine or a pharmaceutically acceptable salt thereof and at least one further therapeutic agent are administered in combined pharmaceutical formulations.

47. A method for the administration of troxacitabine or a pharmaceutically acceptable salt thereof in a host having a tumor, comprising administering an amount of troxacitabine or a pharmaceutically acceptable salt thereof by continuous infusion for a period of at least 72 hours, wherein said amount is sufficient to provide tumor reduction.
